(19) United States
(12) Patent Application Publication
(10) Pub. No.: US 2012/0271404 A1
(43) Pub. Date: Oct. 25, 2012

(54) PATIENT-SPECIFIC MODIFIABLE STENTS

(75) Inventor: Michael J. Levy, Rochester, MN (US)

(73) Assignee: Mayo Foundation for Medical Education and Research, Rochester, MN (US)

(21) Appl. No.: 13/503,502

(22) PCT Filed: Oct. 22, 2010
(86) PCT No.: PCT/US2010/053671

§ 371 Date: Jul. 3, 2012

Related U.S. Application Data
(60) Provisional application No. 61/254,378, filed on Oct. 23, 2009.

(51) Int. Cl. A61F 2/82

(52) U.S. Cl. 623/1.15

(57) ABSTRACT

Implantable stents and stent systems that include one or more features that can be selectively modified at the point of use, i.e., in the surgical suite, etc. The selectively modifiable features may take a variety of forms such as, e.g., length, the presence of flaps, voids, etc.

### RELATED APPLICATION

The present application claims the benefit under 35 U.S.C. §119(e) of U.S. Provisional Patent Application No. 61/254,378, filed on Oct. 23, 2009 and titled PATIENT-SPECIFIC MODIFIABLE STENTS, which is hereby incorporated by reference in its entirety.

Stents and stent systems in which one or more features can be selectively modified as a part of the deployment process are described herein.

Although stents may be used for a variety of blood vessels, stents may also be used in many different body lumens, such as, e.g., the bile duct, ureter, pancreatic duct, vascular locations, esophagus, or any other body lumen.

### SUMMARY OF THE INVENTION

The stents described herein include one or more features that can be selectively modified at the point of use, i.e., in the surgical suite, etc. The selectively modifiable features may take a variety of forms such as, e.g., length, the presence of flaps, voids, etc.

Selectively modifiable stents may provide the user with a product that may better suit the needs of a particular patient while not requiring that the health care facility maintain a large stock of stents that have different combinations of features that may be obtained by point-of-care selective modification.

In one aspect, some embodiments of an implantable stent as described herein include a body comprising a wall; a lumen extending through the body from a first end of the body to a second end of the body, wherein the wall comprises an interior surface facing the lumen and an exterior surface facing away from the lumen, wherein the interior surfaces and the exterior surfaces are constructed of biocompatible materials; and a predetermined line of separation provided in the wall, wherein separating the line of separation forms an opening in the wall between the interior surface of the wall and the exterior surface of the wall.

In some embodiments of an implantable stent as described herein the line of separation comprises a plurality of perforations formed through the wall between the interior surface and the exterior surface.

In some embodiments of an implantable stent as described herein the wall comprises a reduced thickness along the line of separation as compared to a wall thickness on opposite sides of the line of separation.

In some embodiments of an implantable stent as described herein the portion of the wall occupied by the line of separation is formed of a first material, and the portion of the wall that is not occupied by the stent is formed of a second material that is different than the first material. In some embodiments, the first material is soluble in a first solvent and wherein the second material is not appreciably soluble in the first solvent.

In some embodiments of an implantable stent as described herein the line of separation comprises a series of mechanically interlocking fingers distributed along the line of separation.

In some embodiments of an implantable stent as described herein the line of separation comprises a separation element embedded in the wall, wherein removal of the separation element separates the wall along the line of separation. In some embodiments, the separation element comprises a filament embedded in a polymeric material that forms the stent.

In some embodiments of an implantable stent as described herein the line of separation extends around a circumference of the body, wherein separation of the line of separation separates the stent into two discrete smaller bodies, wherein each smaller body of the two discrete smaller bodies comprises a lumen extending through the smaller body from a first end to a second end.

In some embodiments of an implantable stent as described herein the line of separation comprises a shape that creates a flap attached at one end to the body when the wall is separated along the line of separation.

In some embodiments of an implantable stent as described herein the line of separation is in the form of a closed figure such that separation of the line of separation forms a void in the wall of the body in the shape of the closed figure.

In some embodiments of an implantable stent as described herein the stent comprises two or more discrete lines of separation.

In another aspect, some embodiments of an implantable stent system as described herein include a first body and a second body; wherein the first body comprises a wall and a lumen extending through the first body from a first end of the first body to a second end of the first body, wherein the wall comprises an interior surface facing the lumen and an exterior surface facing away from the lumen, wherein the interior surfaces and the exterior surfaces are constructed of biocompatible materials; wherein the second body comprises a wall and a lumen extending through the second body from a first end of the second body to a second end of the second body, wherein the wall comprises an interior surface facing the lumen and an exterior surface facing away from the lumen, wherein the interior surfaces and the exterior surfaces are constructed of biocompatible materials; wherein the second end of the first body comprises external threads on the exterior surface of the wall of the first body and the first end of the second body comprises internal threads on the interior surface of the wall of the second body, wherein the external threads and the internal threads are complementary such that the first body and the second body can be connected by screwing the first body and the second body together using the external threads and the internal threads.

In some embodiments of an implantable stent system as described herein the first end of the first body comprises internal threads on the interior surface of the wall of the first body.

In some embodiments of an implantable stent system as described herein the second end of the second body comprises external threads on the exterior surface of the wall of the second body.

In some embodiments of an implantable stent system as described herein the first end of the first body comprises internal threads on the interior surface of the wall of the first body, and the second end of the second body comprises external threads on the exterior surface of the wall of the second body.

In another aspect, some embodiments of an implantable stent system as described herein include a first body and a second body; wherein the first body comprises a wall and a lumen extending through the first body from a first end of the first body to a second end of the first body, wherein the wall comprises an interior surface facing the lumen and an exterior surface facing away from the lumen, wherein the interior surfaces and the exterior surfaces are constructed of biocompatible materials; wherein the second body comprises a wall and a lumen extending through the second body from a first end of the second body to a second end of the second body, wherein the wall comprises an interior surface facing the lumen and an exterior surface facing away from the lumen, wherein the interior surfaces and the exterior surfaces are constructed of biocompatible materials; wherein the second end of the first body comprises one or more external annular grooves on the exterior surface of the wall of the first body and the first end of the second body comprises one or more internal annular grooves on the interior surface of the wall of the second body, wherein the external annular grooves and the internal annular grooves are complementary such that the first body and the second body can be connected by advancing the external annular grooves of the first body and the internal annular grooves of the second body towards each other.

In some embodiments of an implantable stent system as described herein the second of the first body comprises two or more external annular grooves on the exterior surface of the wall of the first body and the first end of the second body comprises two or more internal annular grooves on the interior surface of the wall of the second body.

In some embodiments of an implantable stent system as described herein the first end of the first body comprises one or more internal annular grooves on the interior surface of the wall of the first body.

In some embodiments of an implantable stent system as described herein the second end of the second body comprises one or more external annular grooves on the exterior surface of the wall of the second body.

In some embodiments of an implantable stent system as described herein the first end of the first body comprises one or more internal annular grooves on the interior surface of the wall of the first body, and the second end of the second body comprises one or more external annular grooves on the exterior surface of the wall of the second body.

In some embodiments of an implantable stent system as described herein the first body comprises a predetermined line of separation provided in the wall, wherein separating the line of separation forms an opening in the wall between the interior surface of the wall and the exterior surface of the wall. In some embodiments, the line of separation comprises a plurality of perforations formed through the wall between the interior surface and the exterior surface. In some embodiments, the wall comprises a reduced thickness along the line of separation as compared to a wall thickness on opposite sides of the line of separation. In some embodiments, the portion of the wall occupied by the line of separation is formed of a first material, and the portion of the wall that is not occupied by the stent is formed of a second material that is different than the first material (in some embodiments, the first material is soluble in a first solvent and wherein the second material is not appreciably soluble in the first solvent). In some embodiments, the line of separation comprises a series of mechanically interlocking fingers distributed along the line of separation. In some embodiments, the line of separation comprises a separation element embedded in the wall, wherein removal of the separation element separates the wall along the line of separation (in some embodiments, the separation element comprises a filament embedded in a polymeric material that forms the stent). In some embodiments, the line of separation extends around a circumference of the first body, wherein separation of the line of separation separates the first body into two discrete smaller bodies, wherein each smaller body of the two discrete smaller bodies comprises a lumen extending through the smaller body from a first end to a second end. In some embodiments, the line of separation comprises a shape that creates a flap attached at one end to the body when the wall is separated along the line of separation. In some embodiments, the line of separation is in the form of a closed figure such that separation of the line of separation forms a void in the wall of the body in the shape of the closed figure. In some embodiments, the first body comprises two or more discrete lines of separation.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a line of separation may be used to refer to one, two, three or more lines of separation.

The term “and/or” means one or all of the listed elements or a combination of any two or more of the listed elements.

The above summary is not intended to describe each embodiment or every implementation of the present invention. Rather, a more complete understanding of the invention will become apparent and appreciated by reference to the following Description of Illustrative Embodiments and claims in view of the accompanying figures of the drawing.

### BRIEF DESCRIPTION OF THE VIEWS OF THE DRAWING

The present invention will be further described with reference to the figures of the drawing, wherein:

FIG. 1 is a perspective view of one embodiment of a stent as described herein.

FIG. 2 is a cross-sectional view of a portion of the stent of FIG. 1 taken along line 2-2 in FIG. 1 depicting one illustrative embodiment of a line of separation.

FIG. 3 depicts another illustrative embodiment of a line of separation in the form of a row if perforations.

FIG. 4 is a cross-sectional view depicting another illustrative embodiment of a line of separation in the form of selectively soluble materials.

FIG. 5 depicts another illustrative embodiment of a line of separation in the form of a row of interlocking mechanical fingers.

FIG. 6 depicts another illustrative embodiment of a line of separation in the foul of a filament embedded in the wall of the stent.

FIGS. 7 & 8 depict an illustrative embodiment of a stent that includes multiple lines of separation configured and arranged to form flaps.

FIGS. 9 & 10 depict another illustrative embodiment of a stent that includes multiple lines of separation configured and arranged to form flaps at an end of the stent.

FIGS. 11 & 12 depict an illustrative embodiment of a stent that includes multiple lines of separation configured and arranged to form voids in the wall of the stent.

FIG. 13 depicts an illustrative embodiment of a stent that includes multiple lines of separation configured and arranged to separate the stent wall such that the length of the stent can be reduced.

FIG. 14 depicts, in a cross-sectional view, an illustrative embodiment of a stent system that includes multiple stent bodies with threaded ends.

FIG. 15 depicts an illustrative embodiment of a stent body that includes ends with annular grooves in a partial cross-sectional view.

### DESCRIPTION OF ILLUSTRATIVE EMBODIMENTS

In the following description of illustrative embodiments, reference is made to the accompanying figures of the drawing which form a part hereof, and in which are shown, by way of illustration, specific embodiments of stents and stent systems. It is to be understood that other embodiments may be utilized and structural changes may be made without departing from the scope of the present invention.

The stents described herein may be manufactured using a wide variety of materials. For those stents that include a line of separation, the material(s) selected for the stent may need to be chosen based on the ability of the material(s) to provide the functionality (e.g., separability) described herein. Reference may be had to the one or more of the following patents for further details regarding potentially suitable techniques for construction, manufacturing, and/or deployment of stents: U.S. Pat. Nos. 4,733,665; 4,739,762; 5,195,984; 5,401,257; 5,725,572; 5,735,871; 5,755,781; 5,853,419; 5,861,027; 6,007,573; 6,059,810; 6,099,561; 6,132,471; 6,200,337; 6,206,916; and 7,338,530; as well as in U.S. Patent Application Publications US 2010/0114325; US 2010/0049328; and US 2008/0249457; etc.

Some materials that may potentially be used to manufacture the stents described herein may include, but are not limited to: SOF-FLE^{X}, a type of polyether urethane, silicone, block co-polymers, urethanes, polyethylene, polystyrene, polytetrafluoroethylene (PTFE), fluorinated ethylene propylene (FEP) and the like, and combinations thereof. Some specific examples may also include TEFLON (a polytetrafluoroethylene) which is discussed for use in stents in U.S. Pat. No. 6,132,471, and PELLETHANE (a biocompatible thermoplastic polyurethane elastomer available from Dow Corning and that has been approved by the Food and Drug Administration for implantation) which is discussed for use with stents in U.S. Pat. No. 7,338,530.

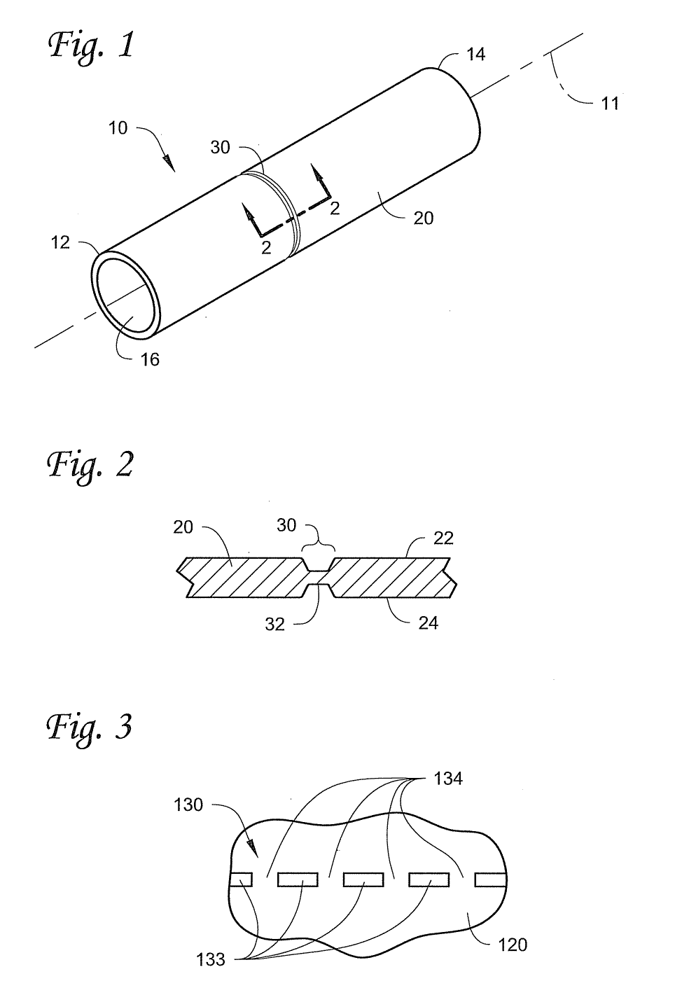

One illustrative embodiment of a stent as described herein is depicted in the perspective view of FIG. 1. The stent includes a body member 10 that has a wall 20 extending between a first end 12 and a second end 14. A longitudinal axis 11 extends between the first end 12 and the second end 14 such that the stent 10 has a length measured along the longitudinal axis 11 between the first end 12 and the second end 14.

The stent 10 includes a lumen 16 that extends through the stent for the first end 12 to the second end 14. The lumen 16 is defined by the wall 20 of the stent 10. The wall 20 includes an interior surface facing the lumen 16 and an exterior surface facing away from the lumen 16. The materials used to construct the interior surfaces and the exterior surfaces of the stents described herein are preferably biocompatible materials. As used herein, “biocompatible materials” are materials that can be implanted within a living body for an extended period of time, e.g., weeks, months and/or years.

The stent 10 of FIG. 1 also includes a predetermined line of separation 30 formed in the wall 20 such that separating the line of separation forms an opening in the wall 20 between the interior surface of the wall 20 and the exterior surface of the wall 20. It may be preferred that the line of separation 30 extend around the perimeter of the stent 10 such that separation of the wall 20 along the line of separation 30 results in complete separation of the stent 10 such that two separate, distinct and smaller stents are formed from the original unitary stent 10

In connection with FIG. 2 (which is a cross-sectional view of the stent 10 of FIG. 1 taken along line 2-2 in FIG. 1), one illustrative embodiment of line of separation 30 in the form of a reduced thickness section 32 of wall 20 is depicted. The reduced thickness of section 32 is relative to the thickness of the wall 20 on opposing sides of the section 32 as depicted in FIG. 2. As a result, when the line of separation n 30 is subjected to separation forces, e.g., tension, tearing, shearing, etc., the wall 20 of the stent 10 preferably separates along the predetermined line defined by line of separation 30. That separation results in an opening formed in the wall 20 between the interior surface 22 of the wall 20 and the exterior surface 24 of the wall 20.

The lines of separation provided in connection with the stents described herein may, in some embodiments, preferably be separable without requiring the use of tools such as blades, scissors, etc. to cut the stent walls. It may be further preferred that, in at least some embodiments, the stent walls be separable along the lines of separation by manipulating the stents to produce tension across the line of separation.

The lines of separation provided in connection with the stents described herein may take many different forms, some illustrative embodiments of which are described in connection with FIGS. 2-6. Although the different embodiments are depicted separately, a stent as described herein may include lines of separation from any two or more of the different types described herein (or even other types not explicitly described herein).

Referring to FIG. 3, another embodiment of a line of separation 130 in the form of a plurality of perforations 133 is depicted. The perforations 133 are, in the depicted embodiment, separated by lands 134. The perorations 133 may preferably be provided in the of openings between the interior surface of the stent wall 120 and the exterior surface of the stent wall 120, although in some embodiments, the perforations 133 may be in the form of thinned regions of the wall 120 (see, e.g., FIG. 2 for an example of a thinned region of a stent wall). The lands 134 between adjacent pairs of perforations may be the same thickness as the portions of the stent wall surrounding the line of separation 130 or they may be somewhat thinner than the surrounding portions of the stent wall 120. The relative length of the perforations and/or lands 134 may be adjusted to provide desired separability of the wall 120 along the line of separation 130.

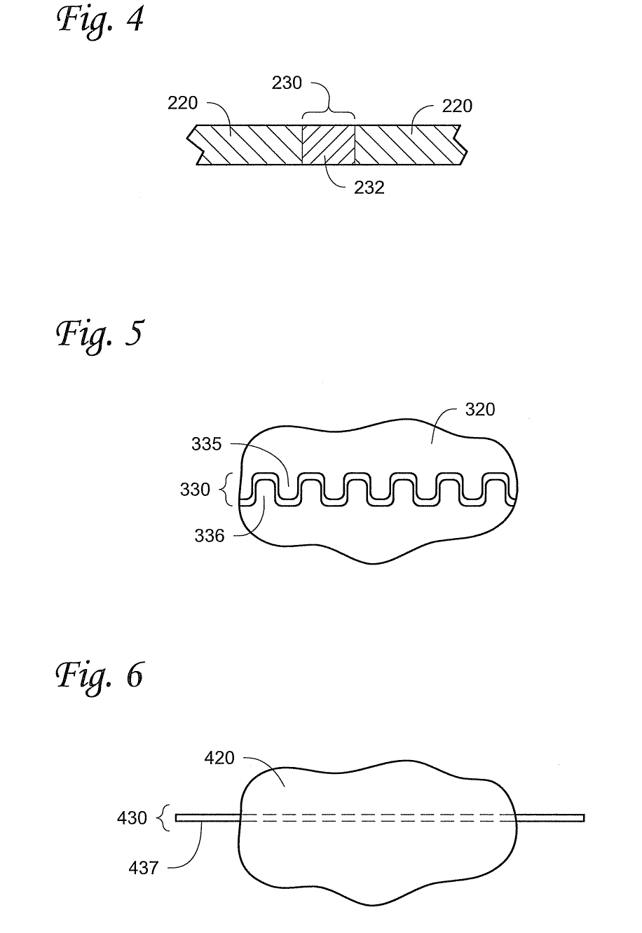

Referring to FIG. 4, another embodiment of line of separation that may be used in connection with the stents described herein is depicted in a cross-sectional view, with the line of separation 230 being formed by a section of a first material 232 that is different from a second material used to form the wall 220 surrounding the line of separation 230. The first material used in section 232 may preferably differ from the surrounding second material in one or more characteristics such that the wall 220 can be separated along the line of separation 230. The different characteristics of the material in section 232 may be, e.g., tensile strength, shear strength, etc.

In at least some embodiments, the first material of the section 232 may be soluble in a first solvent while the second material forming the wall around the line of separation 230 is not appreciably soluble in the same solvent. As used herein, “not appreciably soluble” means that exposure of the second material to the solvent used to promote separation of the first material (under the same conditions) does not weaken the second material to a degree that accurate separation of the stent wall 220 along the line of separation 230. Such differential solubility may be used advantageously to facilitate separation of the wall 220 along the line of separation 230 during and/or after exposure of the section 232 to the solvent.

FIG. 5 depicts another illustrative embodiment of a line of separation 330 that may be used to provide separability of a stent wall 320 as described herein. The line of separation 330 includes a series of mechanically interlocking fingers 335 and 336 that are distributed along the line of separation 330. The interlocking fingers 335 and 336 may alternately extend from the wall 320 on opposite sides of the line of separation 330. Examples of potentially suitable interlocking finger constructions may be seen in, e.g., zipper constructions, dovetail joints, etc.

FIG. 6 depicts another illustrative embodiment of a line of separation 430 in the form of a separation element 437 that may be embedded (partially and/or completely) in the material used to form the stent wall 420. In at least some embodiments, the wall 420 may be made of a polymeric material in which the separation element 437 is embedded. Removal of the separation element 437 may preferably result in separation of the wall 420 along the line of separation 430 and/or may provide an are of reduced thickness that can then be separated (see, e.g., FIG. 2). The separation element 437 may be in the form of a filament, wire, etc. that has sufficient tensile strength to accomplish separation of the stent wall 420 during its removal.

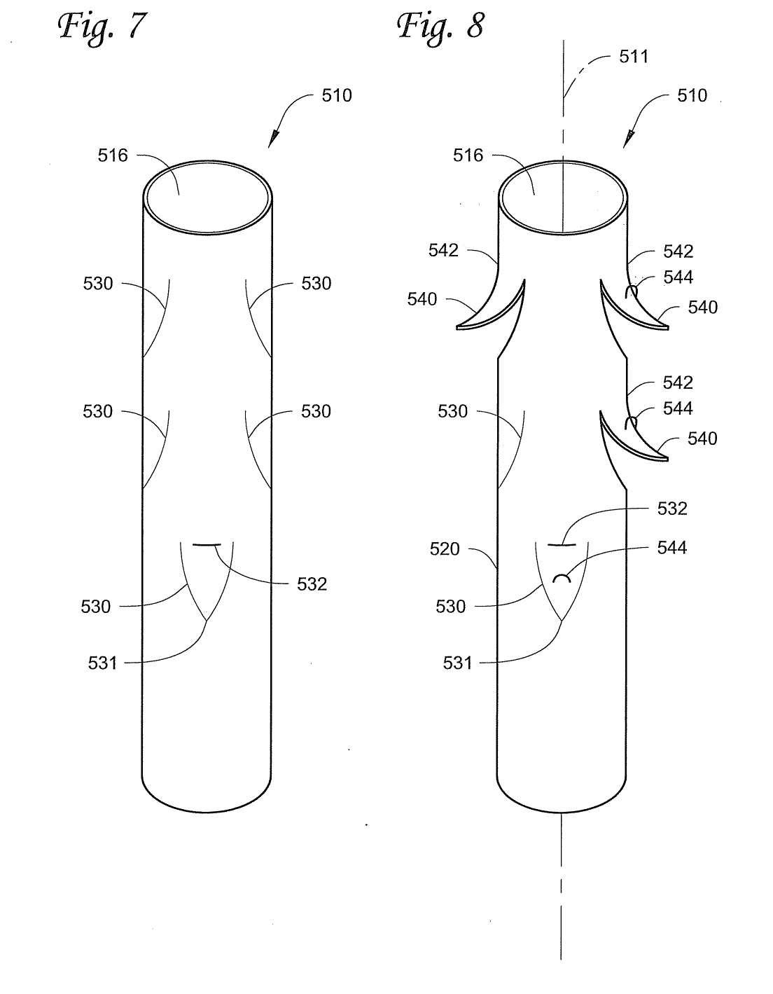

The lines of separation provided in the stents described herein may take a variety of different configurations or shapes, some illustrative examples of which are depicted in FIGS. 7-13. The stent 510 depicted in FIGS. 7 and 8 includes multiple discrete lines of separation 530 that can be separated to form flaps 540 that are connected at a base 542 to the wall 520 of the stent 510. The flaps 540 extending outwardly as depicted in FIG. 8 may, in some embodiments, provide some resistance to spontaneous migration (proximally and/or distally) of the stent 510 within a body lumen in which the stent 510 is deployed. Although the flaps 540 are all shown as extending outwardly in the same direction (relative to longitudinal axis 511), in other embodiments, some of the flaps 540 may extend in the opposite direction or any other direction. In another alternative, although the flaps 540 are depicted in FIG. 8 as extending outwardly from the lumen 516, some or all of the flaps may extend inwardly into the lumen 516 if so desired.

In still other embodiments, portions of the stent could be made stiffer than surrounding portions (by, e.g., the addition of an extra layer of material, etc.) to assist in the separation of the stent walls. For example, the tip regions of the flaps 540 depicted in FIG. 8 could be modified such that the portion located proximate the tip 531 formed by the line of separation 530 was stiffer than the portion of the stent 510 located outside of the line of separation 530 (or vice versa). The difference in stiffness on the opposite sides of the line of separation 530 may potentially cause the stent wall to separate more readily along the lines of separation (see FIG. 7).

Yet another optional feature is depicted in FIG. 7, where the stent 510 may include a slit 532 formed through the wall of the stent 510 at the base of the flap 540 formed by separation of the stent wall along the line of separation 530. The slit 532 may take any of a number different forms, e.g., a true slit, a slot, etc. The placement of such a slit 532 may predispose the flap 540 to protrude outwardly from the stent 510 when the line of separation 530 is separated as described herein. In some embodiments, any such slit 532 may preferably not extend completely across base of flap 540 so that the flap 540 does not completely separate from the stent 510 when the line of separation 530 is separated.

Another optional feature that is depicted in connection with the embodiment of stent 510 in FIG. 8 is the inclusion of a separation handle such as a loop, hook, opening or other handle that provides a location where the wall of the stent 510 can be grasped to provide a force that assists in separating the wall of the stent 510 along one or more lines of separation. In the embodiment depicted in FIG. 8, the separation feature 544 is in the form of a loop, but it could be replaced by any other feature that provides a location where the wall of the stent 510 can be grasped.

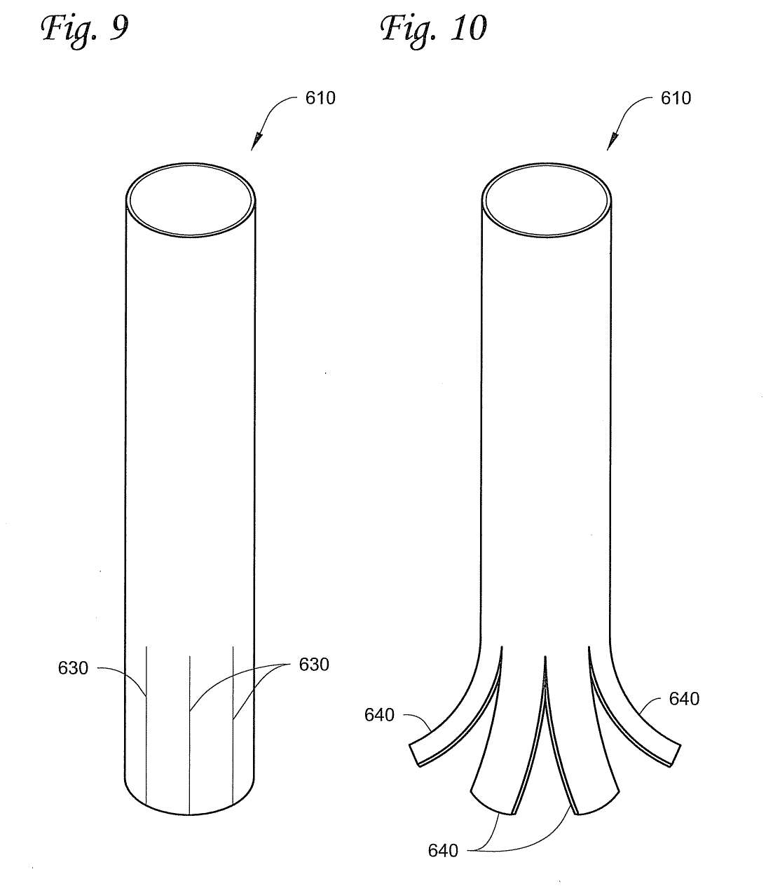

The stent 610 depicted in FIGS. 9 and 10 includes lines of separation 630 that are not contained entirely within the wall of the stent 610 (in contrast to, e.g., the lines of separation 530 depicted in stent 510 of FIGS. 7 and 8). The lines of separation 630 extend inwardly from an end along the length of the stent 610. Separation of the lines of separation 630 may create flaps 640 connected to the stent 610 at one end. The flaps 640 may, in some embodiments, inhibit spontaneous migration (proximally and/or distally) of the stent 610 in a body lumen in which the stent 610 is deployed.

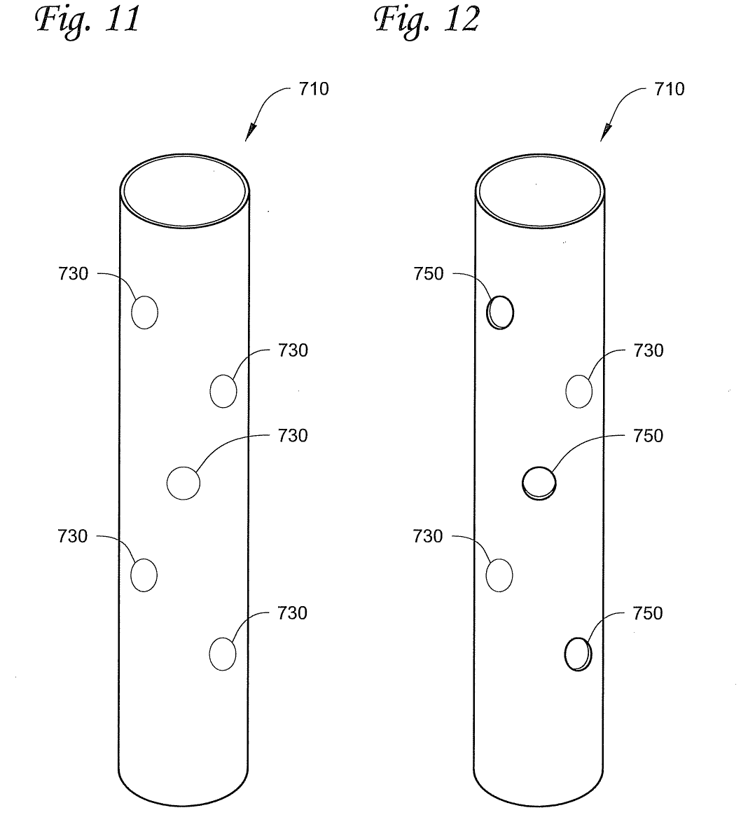

The stent 710 depicted in FIGS. 11 and 12 includes lines of separation 730 that are in the form of closed figure. In other words, the lines of separation 730 do not include an end. As a result, separation of the stent wall along the lines of separation 730 forms a void or opening in the wall of the stent 710 that is preferably in the shape of the closed figure formed by the line of separation 730. Referring to FIG. 12, the stent 710 includes voids or openings 750 that are left after separation of the lines of separation 730 seen in FIG. 11. Although the lines of separation 730 are all depicted in the of closed figures with the same size and shape, stents as described herein may include different lines of separation that form closed figures that differ in shape and/or size in the same stent.

In connection with the stent 710 of FIGS. 11 and 12, any resulting voids or openings in the wall formed by separation along the lines of separation 730 may facilitate the passage of fluids and other materials through the stent 710. For example, the openings or voids may facilitate drainage of pancreatic side ducts if the stent is located in the pancreatic duct. As another example, the voids or openings may facilitate drainage of the biliary tree (cystic ducts and/or intrahepatic ducts) if the stent is positioned in a bile duct. In the absence of the voids or openings, pancreatic and/or bile fluid flow and drainage may be inhibited, which could potentially increase the risk of pancreatitis or biliary stasis and cholecystitis, jaundice, undrained segments, and cholangitis.

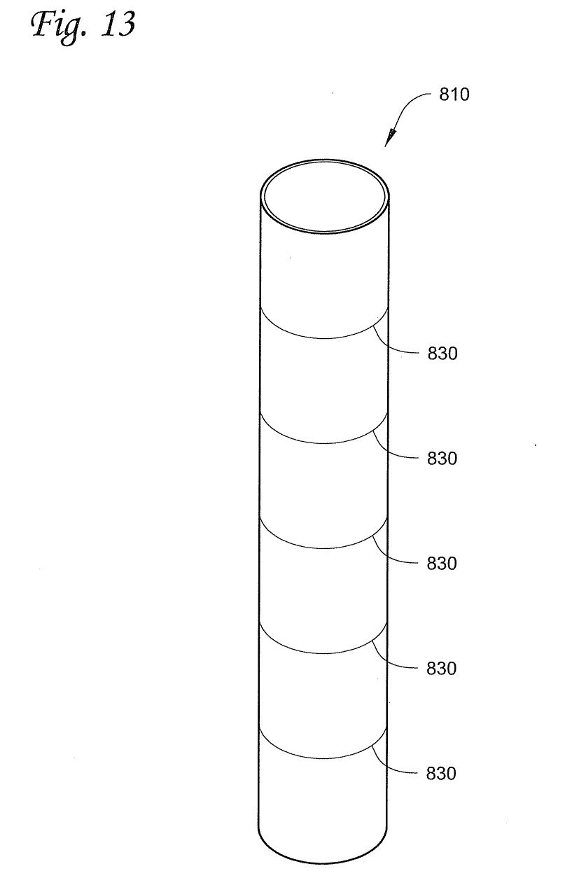

FIG. 13 depicts yet another variation in the lines of separation that may be provided in connection with the stents as described herein. The stent 810 includes lines of separation 830 that extend around the circumference of the body of the stent 810, such that separation of any one of the lines of separation 830 separates the stent 810 into two shorter discrete stent bodies. Although multiple lines of separation 830 are depicted in FIG. 13, the stents described herein may include as few as one circumferential line of separation.

Although different variations in the construction, configuration, etc. of the lines of separation that may be provided in the stents described herein may be illustrated individually in FIGS. 1-13, the stents described herein may include lines of separation that combine the constructions and/or configurations as needed to provide a stent having selected characteristics. For example, different lines of separation in the same stent may be constructed differently (or, in some embodiments, two or more different constructions may be combined to form the same line of separation in a stent). In another example, the same stent may combine lines of separation that form flaps, form voids, and/or change the length of the stent.

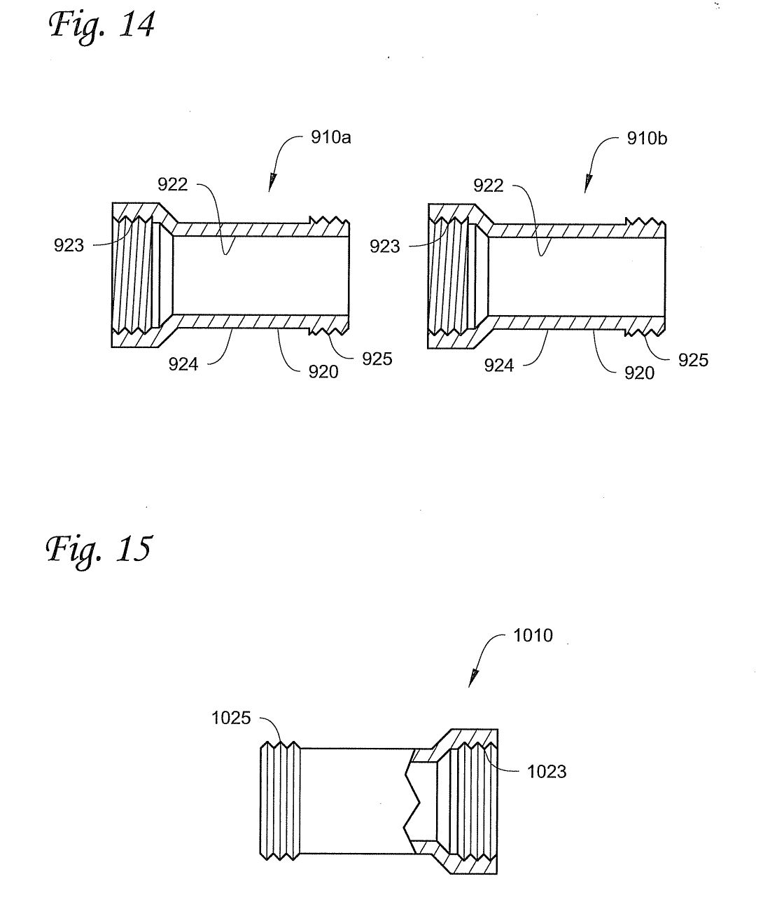

The stents as described herein may also include multiple bodies that can be connected along lines of separation that are formed by, e.g., complementary threads such that two or more stent bodies can be connected by threading them together. FIG. 14 depicts, in a cross-sectional view, one illustrative embodiment of a stent system that includes two stent bodies 910a and 910b (collectively referred to below as bodies 910 for simplicity). Each of the bodies 910 include a stent wall 920 with internal threads 923 located on the interior surface 922 the wall 920 at one end of the body 910 and external threads 925 on the exterior surface 924 of the wall 920 at the opposite end of the body 910. The bodies may be slightly flared to facilitate connection of the bodies 910 by threading the internal threads of one body 910 with the external threads 925 of the other body.

Although only two bodies 910a and 910b are depicted in FIG. 14, the stents system may include three or more different bodies in some embodiments. In another variation, only one end of a stent body may be threaded (externally and/or internally) in some embodiments. In still another variation, the same stent body may include two ends with internal threads or two ends with external threads. Further still, the lines of separation described in connection with FIGS. 1-13 may be provided on any of the bodies in a stent system such as that depicted and described in connection with FIG. 14.

Yet another variation of the stent system depicted in FIG. 14 is depicted in FIG. 15. The stent system depicted in FIG. 15 may preferably be, in all respects other than the threads, that same as the stent system described in connection with FIG. 14. In place of the complementary threads that are connected by rotating the bodies 910 as described above, the stent body depicted in FIG. 15 includes internal annular grooves 1023 on one end of the body 1010 and external annular grooves 1025 on the opposite end of the body 1010. The grooves are annular, not helical, and, thus, do not function as threads. Two stent bodies 1010 may, however, be connected by forcing the annular grooves together, such that one set of internal annular grooves mates with one set of external annular grooves. Although multiple annular grooves are depicted at each end of the body 1010, as few as one groove may be sufficient to provide the desired connection between stent bodies in some embodiments.

In still other embodiments that may provide for separation of stent body into two or more segments, a first stent body may include internal grooves or other depressions while a second stent body includes one or more balls or other structures resiliently supported such that the internal grooves or other depressions receive and retain the ball or other structure in a manner similar to ball detent mechanism to retain the stent segments together, but allow for separation if so desired.

In some embodiments, only one end of a stent body may include annular grooves (external and/or internal). In still another variation, the same stent body may include two ends with internal annular grooves or two ends with external annular grooves. Further still, the lines of separation described in connection with FIGS. 1-13 may be provided on any of the bodies in a stent system such as that depicted and described in connection with FIG. 15.

The complete disclosure of the patents, patent documents, and publications cited in the Background, the Description of Illustrative Embodiments, and elsewhere herein are incorporated by reference in their entirety as if each were individually incorporated.

Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An implantable stent comprising:
a body comprising a wall;
a lumen extending through the body from a first end of the body to a second end of the body, wherein the wall comprises an interior surface facing the lumen and an exterior surface facing away from the lumen, wherein the interior surfaces and the exterior surfaces are constructed of biocompatible materials; and
a predetermined line of separation provided in the wall, wherein separating the line of separation fauns an opening in the wall between the interior surface of the wall and the exterior surface of the wall.

2. A stent according to claim 1, wherein the line of separation comprises a plurality of perforations formed through the wall between the interior surface and the exterior surface.

3. A stent according to claim 1, wherein the wall comprises a reduced thickness along the line of separation as compared to a wall thickness on opposite sides of the line of separation.

4. A stent according to claim 1, wherein the portion of the wall occupied by the line of separation is formed of a first material, and wherein the portion of the wall that is not occupied by the stent is formed of a second material that is different than the first material.

5. A stent according to claim 4, wherein the first material is soluble in a first solvent and wherein the second material is not appreciably soluble in the first solvent.

6. A stent according to claim 1, wherein the line of separation comprises a series of mechanically interlocking fingers distributed along the line of separation.

7. A stent according to claim 1, wherein the line of separation comprises a separation element embedded in the wall, wherein removal of the separation element separates the wall along the line of separation.

8. (canceled)

9. A stent according to claim 1, wherein the line of separation extends around a circumference of the body, wherein separation of the line of separation separates the stent into two discrete smaller bodies, wherein each smaller body of the two discrete smaller bodies comprises a lumen extending through the smaller body from a first end to a second end.

10. A stent according to claim 1, wherein the line of separation comprises a shape that creates a flap attached at one end to the body when the wall is separated along the line of separation.

11. A stent according to claim 1, wherein the line of separation is in the form of a closed figure such that separation of the line of separation fauns a void in the wall of the body in the shape of the closed figure.

12. A stent according to claim 1, wherein the stent comprises two or more discrete lines of separation.

13. An implantable stent system comprising a first body and a second body;
wherein the first body comprises a wall and a lumen extending through the first body from a first end of the first body to a second end of the first body, wherein the wall comprises an interior surface facing the lumen and an exterior surface facing away from the lumen, wherein the interior surfaces and the exterior surfaces are constructed of biocompatible materials;
wherein the second body comprises a wall and a lumen extending through the second body from a first end of the second body to a second end of the second body, wherein the wall comprises an interior surface facing the lumen and an exterior surface facing away from the lumen, wherein the interior surfaces and the exterior surfaces are constructed of biocompatible materials;
wherein the second end of the first body comprises external threads on the exterior surface of the wall of the first body and the first end of the second body comprises internal threads on the interior surface of the wall of the second body, wherein the external threads and the internal threads are complementary such that the first body and the second body can be connected by screwing the first body and the second body together using the external threads and the internal threads.

14. A stent system according to claim 13, wherein the first end of the first body comprises internal threads on the interior surface of the wall of the first body.

15. A stent system according to claim 13, wherein the second end of the second body comprises external threads on the exterior surface of the wall of the second body.

16. A stent system according to claim 13, wherein the first end of the first body comprises internal threads on the interior surface of the wall of the first body, and wherein the second end of the second body comprises external threads on the exterior surface of the wall of the second body.

17. An implantable stent system comprising a first body and a second body;
wherein the first body comprises a wall and a lumen extending through the first body from a first end of the first body to a second end of the first body, wherein the wall comprises an interior surface facing the lumen and an exterior surface facing away from the lumen, wherein the interior surfaces and the exterior surfaces are constructed of biocompatible materials;
wherein the second body comprises a wall and a lumen extending through the second body from a first end of the second body to a second end of the second body, wherein the wall comprises an interior surface facing the lumen and an exterior surface facing away from the lumen, wherein the interior surfaces and the exterior surfaces are constructed of biocompatible materials;
wherein the second end of the first body comprises one or more external annular grooves on the exterior surface of the wall of the first body and the first end of the second body comprises one or more internal annular grooves on the interior surface of the wall of the second body, wherein the external annular grooves and the internal annular grooves are complementary such that the first body and the second body can be connected by advancing the external annular grooves of the first body and the internal annular grooves of the second body towards each other.

18. A stent system according to claim 17, wherein the second of the first body comprises two or more external annular grooves on the exterior surface of the wall of the first body and the first end of the second body comprises two or more internal annular grooves on the interior surface of the wall of the second body.

19. A stent system according to claim 17, wherein the first end of the first body comprises one or more internal annular grooves on the interior surface of the wall of the first body.

20. A stent system according to claim 17, wherein the second end of the second body comprises one or more external annular grooves on the exterior surface of the wall of the second body.

21. A stent system according to claim 17, wherein the first end of the first body comprises one or more internal annular grooves on the interior surface of the wall of the first body, and wherein the second end of the second body comprises one or more external annular grooves on the exterior surface of the wall of the second body.

22-33. (canceled)
